(19) United States
(12) Patent Application Publication
(10) Pub. No.: US 2024/0218317 A1
(43) Pub. Date: Jul. 4, 2024

(54) APPLICATION OF BACILLUS VELEZENSIS IN EXTRACTION OF HEPARIN FROM AQUATIC PRODUCT

(71) Applicant: GUANGDONG OCEAN UNIVERSITY, Zhanjiang City, Guangdong Province (CN)

(72) Inventors: Saiyi ZHONG, Zhanjiang City, Guangdong Province (CN); Huanming LIU, Zhanjiang City, Guangdong Province (CN); Shouchun LIU, Zhanjiang City, Guangdong Province (CN); Pengzhi HONG, Zhanjiang City, Guangdong Province (CN); Chujin DENG, Zhanjiang City, Guangdong Province (CN)

(21) Appl. No.: 17/927,699

(22) PCT Filed: Mar. 24, 2022
(86) PCT No.: PCT/CN2022/082791

§ 371 Date: Nov. 24, 2022

(30) Foreign Application Priority Data
Jul. 5, 2021 (CN) 202110754981.9

(51) Int. Cl. C12N 1/20; C12P 19/26; C12R 1/07

(52) U.S. Cl. CPC C12N 1/205; C12P 19/26; C12R 2001/07

(57) ABSTRACT

An application of Bacillus velezensis HL-5 capable of improving an extraction rate of heparin in aquatic products, the Bacillus velezensis HL-5 is preserved in Guangdong Microbial Culture Collection Center on Jul. 2, 2018, with a preservation number of GDMCC No.: 60403, which is applied to extract heparin from shellfish. Compared with a conventional enzymolysis method, the extraction rate of the heparin is increased by 10.55%-72.07%. The Bacillus velezensis HL-5 has a stronger capability of producing lipase and protease, and has a good application prospect in the extraction of the heparin of aquatic products.

### BACKGROUND

### 1. Technical Field

The present disclosure generally relates to the field of microorganisms, and especially relates to a Bacillus velezensis HL-5 capable of improving an extraction rate of heparin from aquatic products and an application thereof.

### 2. Description of Related Art

Heparin is an acidic mucopolysaccharide alternately composed of glucosamine, L-eduraldehyde glycoside, N-acetylglucosamine and D-glucuronic acid, which has a high content of sulfate group, is a known biological macromolecule with the highest negative charge density, has a good anticoagulant effect and is the most widely used anticoagulant in clinic. In recent years, studies have also found that the heparin has effects of reducing blood lipids, anti-inflammatory, anti-atherosclerosis, anti-tumors, inhibiting bacterial adhesion and so on, and is one of important mucopolysaccharide biochemical medicaments.

Generally, the heparin comes from intestinal mucosal extracts of pigs, cattle and sheep, and also exists in blood vessel walls and lungs of animals; however, development of the heparin from terrestrial mammals is limited due to an influence of heparin pollution events, mad cow disease and religious beliefs, some scholars have proposed to find new heparin resources from relatively clean marine organisms to replace the terrestrial animals. The ocean accounts for more than 71 percent of a surface area of the earth, so that a huge biological bank and a unique environment thereof have created abundant biological resources. In recent years, researches for finding alternative raw materials from the marine organisms as a source of the heparin have been increasing, so that it has been reported that the heparin can be extracted from a variety of marine organisms, such as bay scallops, a variety of seaweeds and marine bacteria, shrimps, mangrove crabs, tunas, mussels and clams.

An enzymolysis method is a common method used for extracting the heparin. Protease can destroy glycopeptide bonds that covalently bind the heparin to proteins, thereby the heparin can be separated from heparin-protein complexes, and heteroproteins can also be decomposed into small peptides. Finally, the heparin/heparinoid can be prepared by adjusting a pH value, performing thermal denaturation and salting out to remove enzyme proteins and degraded proteins.

For example, a Chinese Patent CN 201710419627.4 discloses a method for extracting heparin from complex enzyme that the heparin is extracted in an enzymolysis manner by using complex enzyme of alkaline protease and trypsin, extraction solution is concentrated and filtered, adsorbed by resin, eluted, precipitated and dried to obtain crude heparin sodium, and the crude heparin sodium is further refined to obtain high-purity heparin sodium products. Such method can improve an enzymolysis efficiency and the yield of crude heparin sodium by extracting the heparin from the porcine intestine mucosa. Furthermore, another Chinese patent CN 200910071972.9 discloses an extraction, separation and purification method of heparin that the heparin can be separated and purified from the porcine intestine mucosa by a protease hydrolysis method, an ultrasonic-assisted salting out method and an ion exchange resin adsorption method, so that an extraction rate of the heparin in the above methods can reach 230.81 mg/kg, which is 40% higher than that of the conventional method. In addition, another Chinese patent CN 201811378238.2 discloses a shellfish heparin and a preparation method thereof that trypsin and papain are adopted to extract the heparin from shellfish by the enzymolysis method.

It should be pointed out that both the porcine intestine mucosa and the shellfish contain a certain amount of fat combined with proteins, and the above methods are difficult to completely separate fat from proteins. The conventional enzymolysis method only focuses on enzymolysis of proteins and ignores enzymolysis of the fat. The fat prevents deep enzymolysis of proteins by protease, which makes the enzymolysis incomplete and affects the extraction rate of heparinoids. There is no method to be reported for solving this problem in the related art, so that it has important practical significance that inventors of the present disclosure conduct in-depth experimental research on this problem.

### SUMMARY

The technical problems to be solved: in view of the shortcomings of the related art, the present disclosure provides an application of Bacillus velezensis HL-5 capable of improving an extraction rate of heparin in aquatic products which can solve the problem that protein and fat can't be successfully separated in the related art, resulting in a low extraction rate and a high cost of heparin prepared by the conventional enzymolysis method.

Based on the above intention of the present disclosure, the inventor of the present disclosure screened Bacillus that can degrade fat and protein, the Bacillus is determined to be Bacillus velezensis HL-5 by performing biological morphology and molecular identification on the Bacillus, and preserved in Guangdong Microbial Culture Collection Center on Jul. 2, 2018, with a preservation number of GDMCC No.: 60403. Other preservation information of Bacillus velezensis HL-5 can be seen from a preservation survival certificate of Bacillus velezensis HL-5.

The inventor of the present disclosure confirms that Bacillus velezensis HL-5 is a Bacillus that can produce lipase and protease, which can degrade fat and protein at low temperatures. Based on this discovery, the present disclosure requests to protect an application of the Bacillus strain in a preparation of heparin, such strain is Bacillus velezensis HL-5. According to records of the related art, both terrestrial animals and marine animals are sources for extracting heparin. Bacillus velezensis HL-5 of the present disclosure that has been separated and identified can prepare the heparin from marine organisms or terrestrial animals under suitable conditions. As one embodiment that Bacillus velezensis HL-5 is used to extract the heparin, the present disclosure describes in detail steps and methods of extracting the heparin from shellfish flesh (or tissue) in the marine organisms. Based on technical ideas and suggestions of the present disclosure, those skilled in the art can also use the terrestrial animals as a source material of the heparin in combination with conventional technologies to obtain the heparin from the terrestrial animals.

Specifically, the present disclosure gives a detailed description that the Bacillus velezensis HL-5 is inoculated to shellfish homogenate for preparing the heparin by using a fermentation method.

Furthermore, the method of preparing the heparin by the fermentation method includes: cultivating the strain to obtain a seed solution; inoculating the seed solution into shellfish homogenate; culturing the seed solution that has been inoculated into the shellfish homogenate, adding glucose and continuing to culture and ferment; after finishing the fermentation, inactivating enzyme to obtain raw material for preparing the heparin.

Furthermore, the method of preparing the heparin by the fermentation method includes: inoculating the strain into an LB liquid culture medium, and culturing for 24 h in conditions of a temperature of 15° C. and a shaking speed of 150 r/min to obtain a seed solution; collecting bacteria in the seed solution and inoculating the bacteria into the shellfish homogenate, wherein a volume ratio of the seed solution to the shellfish homogenate is 5˜15%; culturing for 4˜12 h at the temperature of 15° C. and the shaking speed of 150 r/min, adding glucose and continuously culturing and fermenting for 30˜40 h, wherein a mass of glucose that has been added is 0.2˜5% of a mass of the shellfish homogenate; and after the fermentation is finished, inactivating the enzyme for 10 min in a boiling water bath, cooling and centrifuging, and taking supernatant as the raw material for extracting and purifying the heparin.

The Bacillus velezensis HL-5 of the present disclosure is separated from soil samples and screened by a tributyrin plate and a skimmed milk plate to obtain a Bacillus that produces lipase and protease. An isolation identification and a performance verification of the Bacillus velezensis HL-5 are described in detail in an embodiment of the present disclosure, what needs to be emphasized in this part is:

primers for identifying the Bacillus velezensis HL-5 are bacterial universal primers: 27F and 1492R; a PCR amplification system for identifying the strain is as follows: 27F 1.5 μL, 1492R 1.5 μL, MightyAmp DNA Polymerase 1.5 μL, 2×MightyAmp Buffer 30 μL, and ddH2O 25.5 μL; a PCR amplification reaction condition is: performing pre-denaturation for 2 min at a temperature of 98° C., performing denaturation for 10 s at the temperature of 98° C., performing renaturation for 15 s at a temperature of 55° C., performing extension for 90 s at a temperature of 68° C., performing extension for 10 min at a temperature of 72° C., and performing 40 cycles from performing denaturation to perform a first extension.

The inventor of the present disclosure further discloses that the Bacillus velezensis HL-5 uses the fat in meat (tissue) as a carbon source to remove the fat in the meat (tissue) and realize a separation of the protein and the fat, so as to promote deep enzymolysis of the protease and the protein, thereby fully releasing the heparin and further improving the extraction rate of the heparin. The present disclosure provides that the Bacillus velezensis HL-5 is used to improve an efficiency of preparing the heparin from the marine organisms, and further, to improve the efficiency of preparing the heparin from the shellfish. Similarly, based on technical ideas and teaching of the present disclosure, it is not difficult for an ordinary skill in the art to combine the conventional technology to figure out that the Bacillus velezensis HL-5 can be used to improve the efficiency of preparing the heparin by taking the terrestrial animals as materials.

Compared with the related art, the present disclosure provides advantages as below:
(1) the present disclosure separates and identifies the Bacillus velezensis HL-5, which enriches strain resources for heparin preparation;
(2) the Bacillus velezensis HL-5 uses the fat in the shellfish as the carbon source, which can remove the fat from the shellfish, promote the deep enzymolysis of the protease and shellfish protein, thereby fully releasing the heparin and improving the extraction rate of the shellfish heparin;
(3) a certain amount of fat is included in an aquatic product, which is combined with the protein. An effective removal of the fat can facilitate a subsequent separation and purification of the heparin;
(4) the Bacillus velezensis HL-5 can also be used for extracting the heparin from other aquatic products, and is further used for extracting the heparin from animals such as pigs, cattle, sheep and other animals, which has a wide range of applications, and has explored a new heparin extraction method.

### BRIEF DESCRIPTION OF THE DRAWINGS

FIG. 1 is a structural schematic view of a phylogenetic tree of Bacillus velezensis HL-5 of the present disclosure.

### DETAILED DESCRIPTION

Reference will now be made in detail to embodiments, examples of which are illustrated in the accompanying drawings. Obviously, implementation embodiments in the description are a part of the present disclosure implementation examples, rather than limiting the protection scope of the present disclosure.

### A First Embodiment

The first embodiment of the present disclosure provides a screening method of the Bacillus velezensis HL-5 and an identification method of the Bacillus velezensis HL-5.

Specifically, a screening method of the Bacillus velezensis hl-5 includes steps as follows:

step (1), weighing 10 g of soil samples and then putting into a 250 ml of conical flask, adding 90 ml of sterile water into the conical flask, shaking, uniformly mixing the conical flask, and boiling the conical flask on an electric stove for 5 min, taking 1 ml of the above mixture into a 50 ml of enrichment medium, to culture for 3 days in conditions of a temperature of 15° C. and a shaking speed of 160 r/min by a constant temperature oscillation mode. Components of the enrichment medium include tributyrin, ammonium sulfate and distilled water. As a preferred embodiment of the present disclosure, the 50 ml of enrichment medium includes 100 μL of tributyrin, 0.5 g of ammonium sulfate and 50 ml of distilled water, which is used after being sterilized for 20 min at a temperature of 121° C.

step (2), taking the medium that has been enriched to perform 10 times dilution on the enrichment medium. Diluting 1 ml of medium that has been enriched in a test tube with 9 ml of sterile water to obtain 10⁻², 10⁻³, 10⁻⁴ and 10⁻⁵ concentration gradients, respectively. Respectively taking 100 μL of sample solution of 10⁻³˜10⁻⁵ concentrations to evenly coat on the tributyrin plate, coating with two plates per gradient, culturing for 2 days in an incubator at a temperature of 15° C., selecting a bacterial colony with a large diameter of a hydrolysis circle, inoculating the bacterial colony on the tributyrin plate by using sterilized toothpicks, and then marking and culturing for 2 days at the temperature of 15° C.

A preparation method of the tributyrin plate is as follows: fully mixing 2 mL of tributyrin, 10 g of peptone, 5 g of yeast powder, 10 g of NaCl, 20 g of agar and 1000 mL of distilled water, and then sterilizing for 20 min at a temperature of 121° C.

step (3), based on a ratio of a diameter of the hydrolysis circle to a diameter of the bacterial colony, selecting the bacterial colony with a large ratio and inoculating the bacterial colony with the large ratio on a nutrient agar plate by a streak plate method, culturing for 2 days at the temperature of 15° C., selecting a single colony and inoculating the single colony on the nutrient agar inclined plane, culturing for 2 days in the incubator with the temperature of 15° C., and then storing in a refrigerator with a temperature of 4° C.

step (4), performing protease production screening on the strains that have been obtained by preliminary screening, that is, inoculating the strains that have been selected by the plate preliminary screening on the skimmed milk plate by using the sterilized toothpicks, marking, and respectively culturing for 48 h in the incubator with the temperature of 15° C., observing a size of the hydrolysis circle around the strains, and comprehensively evaluating capability of the strains for hydrolyzing tributyrin and skimmed milk powder, so as to screen out the strain HL-5.

Specifically, the embodiment provides a method for identifying the strain HL-5. 27F and 1492R bacterial universal primers are used as forward and reverse primers for the identification of the strain HL-5, and MightyAmp DNA Polymerase is used for performing bacterial colony polymerase chain reaction (PCR) amplification.

A PCR amplification system for identifying the strain is as follows: 27F 1.5 μL, 1492R 1.5 μL, MightyAmp DNA Polymerase 1.5 μL, 2×MightyAmp Buffer 30 μL, and ddH₂O 25.5 μL.

A PCR amplification reaction condition is: performing pre-denaturation for 2 min at a temperature of 98° C., performing denaturation for 10 s at the temperature of 98° C., performing renaturation for 15 s at a temperature of 55° C., performing extension for 90 s at a temperature of 68° C., performing extension for 10 min at a temperature of 72° C., and performing 40 cycles from performing denaturation to perform a first extension.

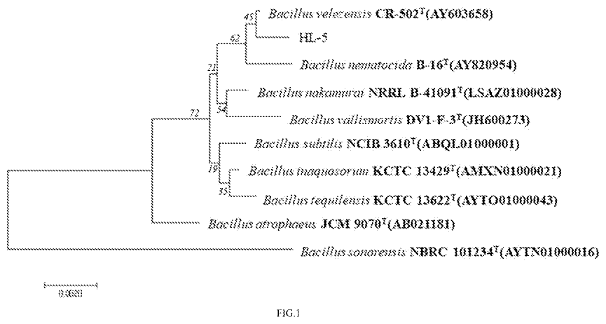

A PCR product is sent to Sangon Biotechnology engineering (Shanghai) Co., Ltd. for sequencing. A sequencing result is performed a 16S rDNA database BLAST search on the sequencing result through NCBI, and a similarity between the strain and Bacillus velezensis CR-502 is the highest, up to 99.78%. Some strains with sequence similarity of 99% are selected, and phylogenetic trees are manufactured by using software Mega5.05 and a maximum likelihood method, results are shown in FIG. 1.

As can be seen from the results in FIG. 1, the strain HL-5 is found to be in the same lineage as the Bacillus velezensis CR-502 on the phylogenetic tree. Based on the above analysis, it can be identified that the strain HL-5 is Bacillus berezensis, which is classified and named as the Bacillus velezensis HL-5.

The nutrient agar plate and the nutrient agar inclined culture medium described in the embodiment are prepared by using the related art, which is not limited in the present disclosure. It should be noted that the enrichment medium and the tributyrin plate described in the embodiment of the present disclosure are preferred embodiments, which is not a special limitation on a culture method of the Bacillus velezensis HL-5. Any culture mediums for culturing the Bacillus velezensis HL-5 through the medium described in the embodiment or other equivalent replacement mediums shall fall within the protection scope of the present disclosure.

### A Second Embodiment

The second embodiment shows an application of the Bacillus velezensis HL-5 for extracting the heparin from the shellfish, and an influence on the extraction rate of the heparin can be explored by comparing the fermentation method with an enzymolysis method in the related art.

### (1) The Enzymolysis Method in the Related Art

Cleaning fresh Philippine clams, imperial concubine clams, bay scallops, Panopea taking the whole meat, adding distilled water (a ratio of material to liquid is 1:3), and putting into a high-speed tissue masher for performing homogenization. Autolyzing the homogenate for 5 h in a constant temperature water bath at a temperature of 55° C., and then sequentially adding 0.5% of 2709 alkaline protease and papain for performing enzymolysis; wherein enzymolysis conditions are as follows: a pH value is pH 8, a temperature is 50° C. and a time is 10 h.

The enzymatic hydrolysate is centrifuged after being cooled by performing enzyme inactivation. The supernatant is taken to be added with 0.4 times volume of ethanol, and is performed alcohol precipitation for 24 h, and then the precipitate is redissolved with distilled water, so that insoluble substances are removed by performing centrifugation. Taking the supernatant and adding a Sevag reagent (V chloroform:V n-butanol=4:1) into the supernatant for performing deproteinization and centrifugation, and taking the supernatant for performing dialysis for 48 h. Performing freeze drying treatment on a sample solution after the sample solution is concentrated, so as to finally obtain eight kinds of crude shellfish heparin, and weighing a mass of each kind of heparin crude product.

### (2) The Fermentation Method of the Present Disclosure

Inoculating the Bacillus velezensis HL-5 preserved on the inclined plane into the LB liquid medium, and culturing for 24 h in conditions of the temperature of 15° C. and the shaking speed of 150 r/min, so as to obtain a Bacillus velezensis HL-5 seed solution for standby.

Cleaning fresh Philippine clams, imperial concubine clams, bay scallops, Panopea taking the whole meat, adding the sterile water (a ratio of material to liquid is 1:3), and putting into the high-speed tissue masher for performing homogenization.

Adding 100 mL of homogenate into the triangular flask that has been sterilized and cooled; taking 8 mL of Bacillus velezensis HL-5 seed solution, performing centrifugation for 10 min at a shaking speed of 8000 r/min, and collecting a bacteria, adding 8 mL of distilled water to wash the bacteria, and further performing centrifugation for 10 min at the shaking speed of 8000 r/min, and collecting the bacteria again; adding the collected bacteria to the triangular flask that includes the shellfish homogenate, culturing for 8 h in conditions of the temperature of 15° C. and the shaking speed of 150 r/min, and then adding 2 g of glucose to the triangular flask for further culturing for 30 h.

after the fermentation is finished, inactivating the enzyme for 10 min in the boiling water bath, cooling the enzyme and then performing centrifugation for 20 min at the shaking speed of 8000 r/min, and taking the supernatant. The supernatant is taken to be added with 0.4 times volume of ethanol, and is performed alcohol precipitation for 24 h, and then the precipitate is redissolved with distilled water, so that insoluble substances are removed by performing centrifugation. Taking the supernatant and adding the Sevag reagent (V chloroform:V n-butanol=4:1) into the supernatant for performing deproteinization and centrifugation, and taking the supernatant for performing dialysis for 48 h. Performing freeze drying treatment on a sample solution after the sample solution is concentrated, so as to finally obtain eight kinds of crude shellfish heparin, and weighing a mass of each kind of heparin crude product.

Dissolving the above crude products of various shellfish heparin prepared by enzymolysis and fermentation methods into water. Determining heparin content in the crude products of various shellfish heparin by taking heparin sodium as a standard substance, and calculating the extraction rate of various shellfish heparins. The extraction rates of several shellfish heparins extracted by the enzymolysis method and the fermentation method are shown in table 1.

TABLE 1
Comparison results of extraction rates of shellfish heparin extracted
by the enzymolysis method and the fermentation method

| | Extraction rate (mg/kg) | | Relative |
|---|---|---|---|
| Name of | Enzymolysis | Fermentation | effect |
| shellfish | method | method | (%) |
| Philippine clam | 238.81 | 296.25 | 24.05 |
| Imperial concubine | 57.80 | 99.82 | 72.70 |
| clam | | | |
| Bay scallop | 180.77 | 242.36 | 34.07 |
| Panopea abrupta | 206.15 | 266.43 | 29.24 |
| Arca Subcrenata | 288.18 | 336.41 | 16.74 |
| White clam | 372.94 | 412.29 | 10.55 |
| Razor clam | 357.19 | 401.18 | 12.32 |
| Decorated clam | 243.90 | 299.28 | 22.71 |
| Average value | 243.22 | 294.25 | 27.80 |

As can be seen from table 1 that compared with the enzymolysis method, the extraction rates of several kinds of shellfish heparins are significantly improved by using the Bacillus velezensis HL-5 fermentation method, wherein the highest extraction rate is increased by 72.70%, the lowest extraction rate is increased by 10.55%, and an average extraction rate is increased by 27.80%, thereby effect is very obvious.

Although the features and elements of the present disclosure are described as embodiments in particular combinations, each feature or element can be used alone or in other various combinations within the principles of the present disclosure to the full extent indicated by the broad general meaning of the terms in which the appended claims are expressed. Any variation or replacement made by one of ordinary skill in the related art without departing from the spirit of the present disclosure shall fall within the protection scope of the present disclosure. Therefore, the protection scope of the present disclosure shall be subject to the protection scope of the claims.

What is claimed is:

1. A strain of Bacillus velezensis HL-5 is characterized in that the Bacillus velezensis HL-5 is preserved in Guangdong Microbial Culture Collection Center, and a preservation number of GDMCC No. is 60403.

2. An application of the Bacillus velezensis HL-5 for extracting heparin from shellfish as claimed in claim 1, comprising the following steps:
step (1), inoculating the Bacillus velezensis HL-5 into an LB liquid culture medium, and culturing for 24 h in conditions of a temperature of 15° C. and a shaking speed of 150 r/min, to obtain a seed solution;
step (2), collecting a whole meat after shelling the shellfish, homogenizing the shellfish meat with sterile water according to that a mass ratio of the shellfish meat to the sterile water is 1:0.5˜1:10, and adding shellfish homogenate that has been obtained into a sterile triangular flask;
step (3), measuring the seed solution of the Bacillus velezensis HL-5 that has been prepared in the step (1) according to that a volume ratio of the seed solution to the shellfish homogenate in the triangular flask is 5%-15%, performing centrifugation and collecting a bacteria, and adding distilled water to resuspend to a volume of an original seed solution, and then performing centrifugation and collecting the bacteria again, and inoculating the bacteria that has been collected for a second time into the triangular flask that has been added with the shellfish homogenate in the step (2);
step (4), culturing the triangular flask that has been inoculated for 4˜12 h in conditions of the temperature of 15° C. and the shaking speed of 150 r/min, and then adding glucose into the triangular flask to continue culturing for 30˜40 h, wherein a mass of the glucose that has been added is 0.2˜5% of a mass of the shellfish homogenate;
step (5), after the fermentation is finished, inactivating enzyme for 10 min in a boiling water bath, cooling and centrifuging, and taking supernatant as a raw material for subsequently extracting and purifying the heparin.

3. The application as claimed in claim 2, wherein the Bacillus velezensis HL-5 is used to improve an extraction rate of the heparin from the shellfish.
